(19) United States
(12) Patent Application Publication
(10) Pub. No.: US 2016/0194573 A1
(43) Pub. Date: Jul. 7, 2016

(54) PROCESS FOR PRODUCING A SUBSTITUTE NATURAL GAS

(71) Applicants: ADVANCED PLASMA POWER LIMITED, Swindon Wiltshire (GB); PROGRESSIVE ENERGY LIMITED, Stroud Gloucestershire (GB)

(72) Inventors: Chris Chapman, Kempsford (GB); Richard Taylor, St. Dennis (GB); Phillip Cozens, Soulbury (GB); Massimiliano Materazzi, London (GB); Chris Manson-Whitton, Stroud (GB)

(73) Assignees: ADVANCED PLASMA POWER LIMITED, Swindon (GB); PROGRESSIVE ENERGY LIMITED, Stroud (GB)

(21) Appl. No.: 14/907,738

(22) PCT Filed: Jul. 28, 2014
(86) PCT No.: PCT/GB2014/052305

§ 371 Date: Jan. 26, 2016

(30) Foreign Application Priority Data
Jul. 26, 2013 (GB) 1313402.8

(51) Int. Cl. C10L 3/08; C10J 3/82; F02C 3/20; C07C 1/12; C07C 2/76; C10K 3/04; C10L 3/10

(52) U.S. Cl. CPC C10L 3/08; C10K 3/04; C10J 3/82; C10L 3/104; C07C 1/12; C07C 2/76; F02C 3/20; C10J 2300/165; C10J 2300/0946; C10J 2300/0916; C10J 2300/1659; C10L 2290/04; C10L 2290/24; C10L 2290/42; C10L 2290/38

(57) ABSTRACT

A process for producing a substitute natural gas, the process comprising the steps of providing a synthesis gas comprising hydrogen and carbon monoxide; subjecting the synthesis gas to a water-gas-shift reaction to increase the ratio of hydrogen to carbon monoxide thereby forming a hydrogen-enriched synthesis gas; subjecting the hydrogen-enriched synthesis gas to a methanation reaction to convert at least a portion of the gas into methane thereby forming a methane-enriched gas; and recovering from the methane-enriched gas a methane-containing gas having a Wobbe number of from 43 to 57 MJ/m³.

### REFERENCE TO RELATED APPLICATIONS

This is a US national stage application of PCT/GB2014/052305 filed Jul. 28, 2014 claiming priority to GB 1313402.8 filed Jul. 26, 2013, the entire disclosures of which are expressly incorporated herein by reference.

### FIELD OF THE INVENTION

The invention relates to a process for producing a substitute natural gas.

### BACKGROUND

Substitute natural gas (SNG) can be produced from fossil fuels such as coal, and it is known to incorporate SNG together with natural gas in a gas grid. Substitute natural gas obtained from biofuels is also known and is termed bio-SNG. In view of the need to employ more renewable sources of energy, it is proposed to distribute SNG and bio-SNG together with natural gas in a gas grid.

Renewable bio-SNG may be derived from wet wastes via anaerobic digestion, but insufficient bio-resources are available to provide sufficient renewable gas from this source alone. Therefore, it is necessary to develop an alternative pathway to manufacture renewable bio-SNG from non-digestible biogenic waste sources via, for example, thermal gasification.

In order for a bio-SNG to be incorporated into a gas grid together with natural gas, the bio-SNG will need to exhibit similar properties to that of natural gas—for example comparable levels of impurities and comparable combustion energy outputs. Although methane synthesis from syngas produced from the gasification of solid fuels is known, the process designs that have been developed to date have been predominantly for coal where high throughputs are needed to obtain the required economies of scale. Bio-SNG production from biogenic fuels will require facilities of greatly reduced scale where a different approach is required regarding both the design and operation in order to attain an effective techno-economic solution. For example, the cleaning of bio-syngas to the ppb levels required for catalytic conversion of syngas will be different from a syngas produced from coal or other fossil fuels due to variances in the type and concentration of impurities present. In comparison to syngas derived from coal, syngas derived from biomass for example contains lower levels of sulphur and carbon monoxide, but higher levels of nitrogen and carbon dioxide.

### SUMMARY OF THE INVENTION

The present invention seeks to tackle at least some of the constraints associated with the prior art when applied to biogenic fuels or fuels derived from biogenic wastes or mixed wastes or at least to provide a commercially acceptable alternative solution thereto.

In one aspect, the present invention provides a process for producing a substitute natural gas, the process comprising the steps of:

providing a synthesis gas comprising hydrogen and carbon monoxide;

subjecting the synthesis gas to a water-gas-shift reaction to increase the ratio of hydrogen to carbon monoxide thereby forming a hydrogen-enriched synthesis gas;

subjecting the hydrogen-enriched synthesis gas to a methanation reaction to convert at least a portion of the gas into methane thereby forming a methane-enriched gas; and

recovering from the methane-enriched gas a methane-containing gas having a Wobbe number of from 43 to 57 MJ/m³

### BRIEF DESCRIPTION OF THE FIGURES

FIG. 1 is flow diagram of a process according to the present invention.

FIG. 2 is a flow diagram of a process according to the present invention.

FIG. 3 is a flow diagram of a process according to the present invention.

FIG. 4 is a flow diagram of a process according to the present invention.

### DETAILED DESCRIPTION OF PREFERRED EMBODIMENTS

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

The term “substitute natural gas” or “SNG” as used herein may encompass a gas comprising primarily methane.

The term “synthesis gas” or “syngas” as used herein may encompass a gas mixture comprising primarily hydrogen and carbon monoxide. It may also comprise gaseous species such as carbon dioxide, water vapour and nitrogen, which would together typically not exceed 30% vol. It may also contain impurities such as, for example, solid particulate and tarry species. The amount of these impurities present will typically not exceed 5% w/w.

The term “water-gas-shift reaction” as used herein may encompass a reaction in which carbon monoxide reacts with water vapour to form carbon dioxide and hydrogen, i.e.

CO_{(g)}+H₂O₍ᵥ₎→CO_{2(g)}+H_{2(g)}

The term “methanation reaction” as used herein may encompass a reaction in which in which the oxides of carbon react with hydrogen to form methane and water, i.e.

CO_{(g)}+3H_{2(g)}→CH_{4(g)}+H₂O_{(g)} 1).

and

CO_{2(g)}+4H_{2(g)}→CH_{4(g)}+2H₂O_{(g)} [The Sabatier reaction] 2).

The term “Wobbe number” as used herein is defined as:

$${I}_{W} = \frac{{V}_{C}}{\sqrt{{G}_{S}}}$$

where I_{W} is the Wobbe number, V_{C} is the higher heating value or higher calorific value, and G_{S} is the specific gravity. The Wobbe number may be calculated by the appropriate methodology such as ISO 6976. The Wobbe number (sometimes referred to as Wobbe index) provides an indication of the interchangeability of fuel gases and is universally used as a determinant in gas quality specifications used in gas network or transportation utilities. In physical terms the Wobbe number compares the combustion energy output of fuel gases of varying composition for an appliance (i.e. boiler or cooker) whereby two fuels having an identical Wobbe number will also have the same energy output (assuming all other factors such as pressure and flow rate are kept constant). The Wobbe number is especially important when considering the impact of injecting SNG into the gas grid.

Unless otherwise specified, any pressure values recited herein are absolute pressures, rather than values relative to atmospheric pressure.

The substitute natural gas produced by the process of the present invention exhibits similar properties to that of natural gas, and is therefore suitable to be combined with natural gas in a gas grid. It may also be suitable for use as a transport fuel, for example as a substitute compressed natural gas (CNG) or liquefied natural gas (LNG).

The inventors have surprisingly found that it is possible to carry out the process of the present invention using synthesis gas derived from waste biomass. Accordingly, the process is capable of producing renewable bio-SNG.

In comparison to processes known in the art, the process of the present invention may be operated efficiently at low pressures—typically less than 20 bar pressure, more typically less than 10 bar pressure, even more typically from 1 to 8 bar pressure. This is particularly advantageous when the synthesis gas is derived from a biomass gasifier, such as a fluidised bed, which will typically operate at similarly low pressures. Accordingly, the required level of compression of the synthesis gas is reduced, resulting in an increase in the energy efficiency of the process.

Advantageously, it is possible to recover energy from the process, for example with the use of heat exchangers, the recovery of steam to drive a turbine, or the recovery of a secondary fuel. This increases the overall energy efficiency of the process. The process is relatively simple in comparison to known processes, and is also capable of producing substitute natural gas of high quality in a single pass. (i.e. without product recirculation through the methanation reactors). Furthermore, the process exhibits high energy efficiency even when used with low throughputs, i.e. in plants of a modest scale. This is particularly important when the process is used on synthesis gas derived from biomass, since such processes are typically carried out on a smaller scale in comparison to processes in which the synthesis gas is derived from fossil fuels such as coal.

The methane-containing gas preferably exhibits a gross calorific value (GCV) of from 35 to 45 MJ/m³, more preferably from 36.9 to 42.3 MJ/m³. Such GCV values are similar to natural gas.

Prior to the water-gas-shift reaction, the synthesis gas may be cleaned in a series of stages to remove various contaminant species that would otherwise poison the downstream catalytic processes. The contaminants that may need to be removed will vary depending on the chemical composition of the feedstock and the conditions under which it is converted into synthesis gas. Typical contaminants include sulphur and chloride species, tars, unsaturated hydrocarbons, heavy metals and particulates. Such contaminant species may be removed, for example, by physical or chemical absorption/adsorption.

The synthesis gas is preferably provided at a pressure of less than 10 bar, more preferably from 1 to 8 bar. This is in contrast to SNG production processes known in the art, which typically operate at higher pressures. Accordingly, the level of power required to compress the syngas is reduced, thereby improving the energy efficiency of the process. In addition, as discussed above, the use of low pressures makes the process particularly suitable for use with syngas derived from the fluid-bed gasification of biomass.

The purpose of the present invention is not to produce pure methane but a gas that is suitable for injection into the gas grid. To this end, the methane-containing gas preferably has a Wobbe number of from 45 to 55 MJ/M³, more preferably from 47.2 to 51.4 MJ/m³. This makes the methane-containing gas more suitable for incorporation into a gas grid.

The heating value of SNG may typically be reduced by naturally occurring inert constituents such as nitrogen and carbon dioxide. In this application a portion of the hydrogen-enriched synthesis gas may be subjected to a catalytic alkane and/or alkene formation reaction to convert at least a portion of the gas into C2 and/or C3 and/or C4 alkanes/alkenes. The presence of these C2 and/or C3 and/or C4 alkanes/alkenes increases the heating value of the substitute natural gas. Accordingly, it is possible to prepare a substitute natural gas matching the heating value of natural gas containing these substances without the need for either extensive removal of inert components (either from the gasifier oxidant, or from the products of methanation) or addition of expensive LPG or similar higher alkane fuel gas. This results in a simplified and less costly process. Reducing the need for absolute removal of inert components is particularly important when the process is carried out on synthesis gas derived from biomass, since such synthesis gas will typically contain higher levels of inert components in comparison to synthesis gas derived from fossil fuels.

Examples of catalysts suitable for use in the alkane/alkene formation reaction include cobalt-containing catalysts and iron-containing catalysts. Suitable catalysts may include for example a combination of: Ce, Cu, Co, Fe, Ni, Mn, Ag, Ru, Ca, Mg or Zn, or may be a composite of two or three cations. Such catalysts are capable of reducing CO to produce a mixture of short chain hydrocarbons. Using such catalysts, the production of C2-C4 alkanes/alkenes as a percentage of total CO conversion can be in the right order of magnitude for increasing the Wobbe number to the level required for natural gas substitution.

The ratio of hydrogen to carbon monoxide is preferably increased to about 3:1 or higher. Increasing the ratio of hydrogen to carbon monoxide promotes the methanation reactions, especially at low pressures (up to 10 bar) and low temperatures (for example from 200 to 450° C.).

Preferably, the pressure of the synthesis gas during the water-gas-shift reaction and/or the gas during the methanation reaction and/or the gas during the alkane/alkene formation reaction is less than 10 bar, preferably from 1 to 8 bar. In the presence of a suitable catalyst the use of pressures below 10 bar, preferably less than 8 bar results in the generation of some C2 and C3 alkanes/alkenes, which will increase the Wobbe number of the final substitute natural gas.

The process may further comprise recovering steam produced by the heat released from the water-gas-shift reaction. Such steam may be used to drive a steam turbine, and thereby generate electricity. Accordingly, the energy efficiency of the process may be increased.

Preferably, the step of subjecting the hydrogen-enriched synthesis gas to a methanation reaction and the step of subjecting the hydrogen-enriched synthesis gas to a short chain alkane and/or alkene formation reaction are conducted in the same reaction vessel with multiple catalysts. This may result in a simplified process. Optionally, a separate stream of the hydrogen enriched synthesis gas may be treated in a separate reactor with appropriate catalysts to produce a fuel gas stream high in short chain alkanes and alkenes which, after appropriate refining, may be blended with the SNG product stream to achieve the required Wobbe number for injection into the gas grid.

The water-gas-shift reaction is preferably carried out at a temperature of from 150 to 400° C. Although in many industrial applications the water-gas-shift reaction typically comprises a two-step process, and is preferably conducted at a temperature of from 300 to 400° C. for the first step (high temperature shift) and a temperature of from 150 to 250° C. for the second step (low temperature shift) in the current invention only a single stage, high temperature shift is required in order to achieve the required ratio of H2:CO of 3:1 or greater. This water-gas-shift reaction is typically carried out in the presence of a catalyst, typically a transition metal catalyst such as, for example, Fe₃O₄ (magnetite).

The conventional water gas shift reaction may be conducted using catalysts which are resilient to high sulphur (H₂S) concentrations (“sour shift”) or those which are intolerant of sulphur (“Sweet shift” where H₂S<100 ppm). In the current invention, when waste biomass sources are employed (including from municipal and commercial and industrial wastes) it has surprisingly been found that H₂S levels in the syngas produced by the gasifier are low, so that a high temperature “sweet shift” will invariably be employed in this case.

In one embodiment, only a portion of the synthesis gas is subjected to a water-gas-shift reaction before being re-combined with the remaining portion prior to methanation. In this case, the water-gas-shift reaction is typically taken to completion. Such an arrangement may be easier to control.

The methanation and/or alkane/alkene formation reaction is preferably conducted at a temperature of from 200 to 450° C. Such temperatures allow flexibility of reactor design which can therefore be operated under isothermal or adiabatic conditions (or a combination of reactors operating in series).

The methanation reaction may be carried out in the presence of, for example, a transition metal catalyst such as, for example, a nickel-containing catalyst or an iron-containing catalyst. An example of a suitable catalyst for the methanation catalyst is a Johnson Matthey commercial methanation catalyst in pellet form—Katalco 11-4m containing 22% Ni (metallic basis). The catalysts may be supported on, for example, alumina, silica or zeolite substrates. The use of zeolite or other catalyst substrates with very small pore sizes may restrict the formation of long chain hydrocarbons, for example hydrocarbons longer than C3.

This invention may also incorporate catalyst substrates developed to operate effectively at the temperatures indicated in the foregoing and with high partial pressures of reagents that have not been diluted by product recirculation.

The methane-containing gas may be recovered using either physical or chemical absorption/adsorption techniques, or using pressure swing adsorption. Pressure swing adsorption is preferred since it may also be used for separating nitrogen, carbon dioxide, and other impurities.

Preferably, the recovery of the methane-containing gas produces an off-gas rich in carbon dioxide. Such an off-gas may be “capture ready” and suitable for future CCS (carbon capture and storage). In addition, the removal of inert carbon dioxide from the methane-containing gas increases the heating value of the methane-containing gas. The off-gas rich in carbon dioxide may also be recovered and used in the process as a purge gas or sealing gas. It may also be used as an oxidising gas in the gasifier.

The methane-containing gas may optionally be recovered by removal of nitrogen from the methane-enriched gas. As with carbon dioxide, the removal of inert nitrogen increases the heating value of the methane-containing gas. The recovered nitrogen may also be used as a purge gas. Removal of nitrogen is particularly advantageous when the process makes use of synthesis gas derived from biomass, since such synthesis gas typically contains higher levels of nitrogen in comparison to synthesis gas derived from fossil fuels.

In this embodiment the use of PSA in, the recovery of the methane-containing gas further comprises the recovery of a secondary fuel gas from the methane-enriched gas, preferably having a net calorific value (NCV) of from 4 to 44 MJ/kg. Recovery of such a secondary fuel increases the energy efficiency of the process. The secondary fuel gas is preferably used in a gas turbine or gas engine.

The process preferably further comprises recovering steam generated by the heat released from the methanation reaction. Such steam may be used, for example, to drive a steam turbine and therefore increase the energy efficiency of the process.

The process preferably further comprises a step of recovering or removal of bulk carbon dioxide from the synthesis gas after subjecting the synthesis gas to the methanation reaction in the first stages of a multi-stage methanation reactor. Bulk carbon dioxide is preferably removed after first stage methanation rather than before methanation since the presence of carbon dioxide in the methanation reaction will absorb the heat generated by in the reaction, thereby limiting the temperature rise and avoiding/reducing the recycling of gas to the methanation reaction. The carbon dioxide may be removed in one or two stages by means of pressure swing adsorption and by the Sabatier reaction. Alternatively, bulk carbon dioxide may be removed by PSA prior to the final stage of methanation undertaken via the Sabatier reaction. This reduces the volume of gas present during the methanation reaction, and provides a method to remove carbon dioxide down to the levels required in gas distribution grids and networks. The synthesis gas may need to be reheated prior to the Sabatier methanation reaction.

In a preferred embodiment, the majority of the carbon dioxide is removed from the synthesis gas using pressure swing absorption prior to subjecting the synthesis gas to the Sabatier reaction. In this embodiment, the process preferably further comprises subjecting the synthesis gas to the Sabatier reaction for removal of the carbon dioxide therefrom. In this embodiment, the carbon dioxide levels of the synthesis gas may be reduced to those required for injection of the SNG product into the gas grid.

The synthesis gas may be produced by the gasification and/or plasma treatment of a feedstock material. The feedstock may be a waste material and/or comprises biomass. As discussed above, the process is particularly effective when used with such a synthesis gas.

The water-gas-shift reaction and/or the methanation reaction may be carried out in a single step. In other words, the process may be carried out without the need to re-circulate the hydrogen-enriched synthesis gas back into the water-gas-shift reactor and/or methane-enriched gas back into the methanation reactor. This may result in a simpler, lower cost and lower energy consuming process.

Preferably the synthesis gas is produced according to the process of EP1896774, the disclosure of which is incorporated herein by reference. This is a very efficient and low pressure process. Preferably, the synthesis gas is produced in a waste treatment process comprising:
(i) a gasification step comprising treating the waste in a gasification unit in the presence of oxygen and steam to produce an offgas and a non-airborne, solid char material; and
(ii) a plasma treatment step comprising subjecting the offgas and the non-airborne, solid char material to a plasma treatment in a plasma treatment unit in the presence of oxygen and, optionally, steam, wherein the plasma treatment unit is separate from the gasification unit.

Preferably, high purity oxygen, derived from a Cryogenic air separation unit, (ASU) may be used, rather than from a Pressure Swing Adsorption ASU, as it will contain low levels of nitrogen and will therefore produce a synthesis gas with correspondingly reduced levels of nitrogen which will reduce or even avoid the requirement for nitrogen separation at the SNG refining stage.

In the waste treatment process the waste may be subjected to a microbial digestion step prior to the gasification step. The gasification may take place in a fluid bed gasification unit.

Preferably the synthesis gas is produced by a method comprising:
(i) thermally treating a feedstock material to produce a synthesis gas; and
(ii) plasma-treating the synthesis gas in a plasma treatment unit in the presence of additional carbon dioxide to produce a refined synthesis gas, wherein the additional carbon dioxide is added to the feedstock material during the thermal treatment and/or to the synthesis gas before plasma treatment and/or introduced in the plasma treatment unit. The presence of carbon dioxide helps to maintain the seals on the thermal treatment unit and plasma treatment unit, thereby reducing the addition of oxygen or air into the system, which may disrupt the reactions occurring in the treatment units. It also avoids the introduction of inert diluents, such as nitrogen, which may lower the calorific value of the synthesis gas. Carbon dioxide may also act as an oxidant during gasification. The carbon dioxide added to the feedstock may be derived from the off-gas rich in carbon dioxide recovered from the methane-containing gas. In other words, the off-gas rich in carbon dioxide may be re-cycled back into thermal treatment and/or plasma treatment units.

The process may further comprise combusting the substitute natural gas as a fuel, optionally in combination with at least a portion of natural gas.

In a further aspect, the present invention comprises a substitute natural gas obtainable using the process described herein.

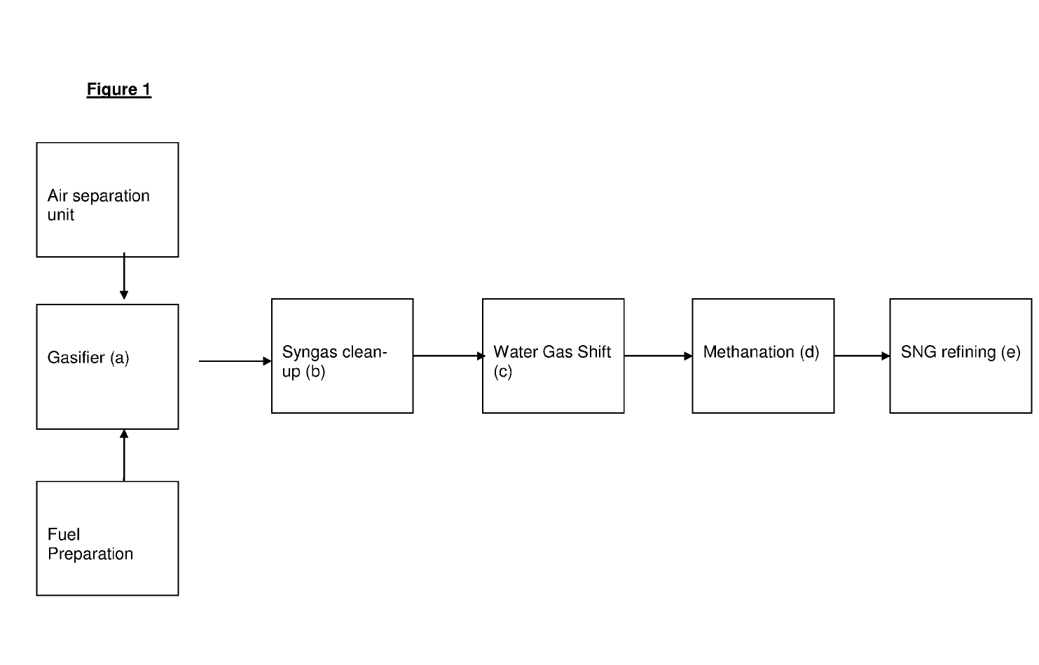

Referring to FIG. 1, in the gasifier (a), the carbonaceous solid feed is converted to a synthesis gas using oxygen and steam as the gasification medium. The type of gasifier (e.g. fluid bed, entrained flow, updraft, plasma) and the nature of the fuel and fuel to oxidant levels employed will impact the quality of the syngas produced. In general, high energy density and friable fuels like coal can be pulverised and fed to an entrained flow gasifier which may be operated at high temperatures (i.e. >1200° C.) to produce a syngas with low levels of tars and gaseous hydrocarbons. In contrast, biomass-containing fuels are of lower heating values and frequently contain inorganic components in the ash (i.e. soda and potash) which are prone to form low melting point eutectic phases. Waste materials in particular, are heterogeneous in nature, and not amenable to being pulverised. For these types of fuels, fluid bed gasifiers are frequently employed due to their ability to handle relatively coarse and chemically heterogeneous materials. These reactors need to be operated at lower temperatures to prevent sintering of the sand causing de-fluidisation of the bed and consequently produce a syngas containing high levels of condensable tars and gaseous hydrocarbon species which can be problematic in the subsequent catalytic water-gas-shift and methanation process stages.

Syngas clean-up (b) is done in a series of stages to remove the various contaminant species that would otherwise poison the downstream catalytic processes. The contaminants that must be removed will vary depending on the chemical composition of the feedstock and the operating conditions within the gasifier but will include sulphur and chloride species, tars and unsaturated hydrocarbons, heavy metals and particulates.

The water-gas-shift reaction (c) is an exothermic catalytic reaction where CO is reacted with steam to produce hydrogen and CO₂.

H₂O(g)+CO(g)→H₂(g)+CO₂(g)

The purpose is to increase the hydrogen to CO ratio to give the molecular concentrations of hydrogen needed at the methanation stage.

The catalytic methanation reaction (d) is highly exothermic with the CO reacting with H₂ to form CH₄ and water according to:

3H₂(g)+CO(g)→CH₄(g)+H₂O(g)

Additionally, methanation is possible through the reaction of hydrogen and CO₂, the Sabatier reaction, especially at high CO₂ and low CO concentrations:

4H₂(g)+CO₂(g)→CH₄(g)+2H₂O(g)

There are a number of different reactor design configurations and catalyst materials that may be applied, depending on the specific process chemistry and thermal rating of the facility.

In the SNG refining stage (e) (methane-separation stage) the methane is upgraded using either physical or chemical liquid absorption techniques or Pressure Swing Adsorption (PSA). The liquid absorption technologies may be used for removal of CO₂ from the product stream. PSA may additionally be used for separating nitrogen and other impurities from the gas. In many coal SNG applications the CO₂ separation is conducted prior to the methanation stage. Additional stages including for example methanation of residual CO₂ by the Sabatier reaction may be required to ensure the gas is of sufficient quality for injection into the distribution grid.

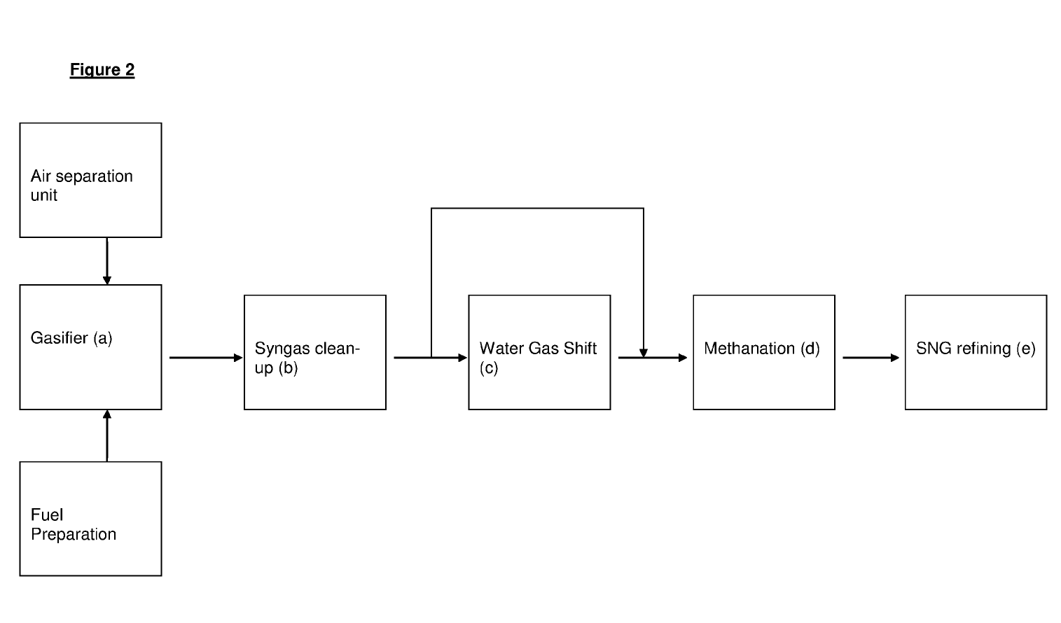

FIG. 2 shows a schematic of a similar process to that shown in FIG. 1. However, in this case, after syngas clean-up (b), a side stream of the gas is subjected to a substantially complete water-gas-shift (c) before being re-combined with the other part of the stream prior to methanation (d).

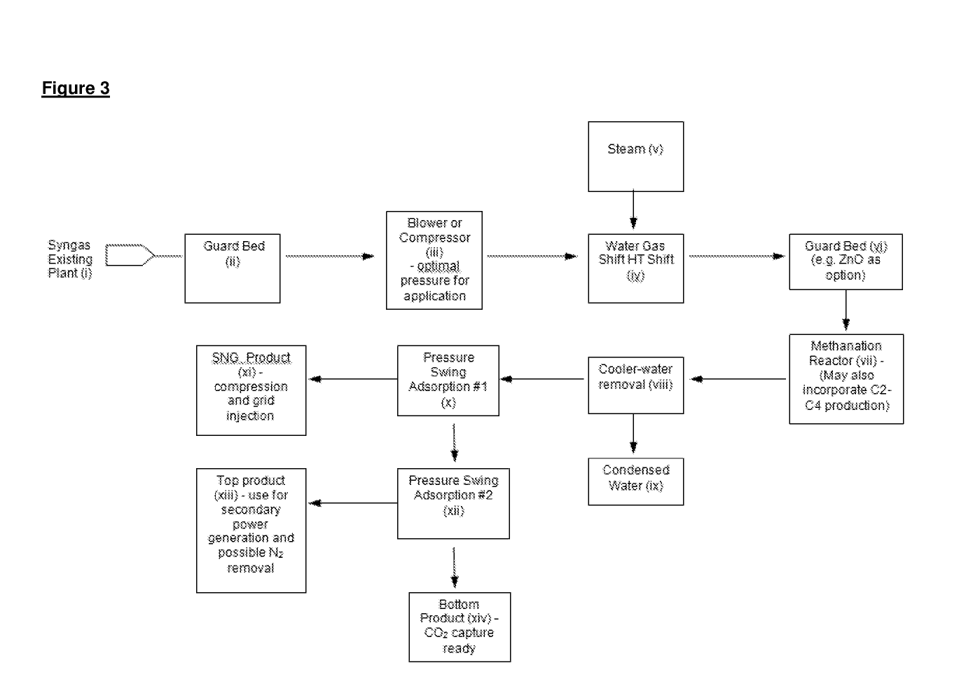

Referring to FIG. 3, syngas from a gasification/plasma treatment unit (i) is passed to a guard bed (ii) for clean-up. The syngas is then compressed to the desired pressure in the compressor (iii) before being passed to the water-gas-shift unit (iv). Steam (v) is added to the reactor and reacts with some of the carbon monoxide in the syngas to produce hydrogen and carbon dioxide, thus increasing the hydrogen to carbon monoxide ratio of the syngas. The resulting hydrogen-enriched syngas is subjected to a further clean-up stage in guard bed (vi) (containing, for example, ZnO) before being passed to methanation reactor (vii). In methanation reactor (vii), hydrogen and carbon monoxide in the syngas is converted to methane and water. With use of appropriate catalysts C2-C4 alkanes/alkenes may also be produced in the methanation reactor. The resulting methane-enriched gas is then subjected to cooling and water removal (viii) to separate condensed water generated in the methanation reaction (ix) The bulk of the water is removed as condensate resulting from the cooling of the gas stream. The moisture level can be further reduced to the levels required for injection into the grid by an appropriate technique such as a dedicated PSA unit or using a desiccant before being passed to a first pressure swing adsorption unit (x). The first pressure swing adsorption unit produces a top product of methane-containing gas having a Wobbe number of from 43 to 57 MJ/m³ (xi) (substitute natural gas). This substitute natural gas is then compressed for injection into a gas grid. The bottom product is passed to a second pressure swing adsorption unit (xii), which produces a top product (xiii) of a secondary fuel gas having a net calorific value (NCV) of from 4 to 44 MJ/kg and a bottom product (xiv) rich in carbon dioxide. The top product (xiii), following optional nitrogen removal, may be used for secondary power generation, thereby compensating for the parasitic load of the process. The bottom product (xiv) is carbon dioxide “capture ready”.

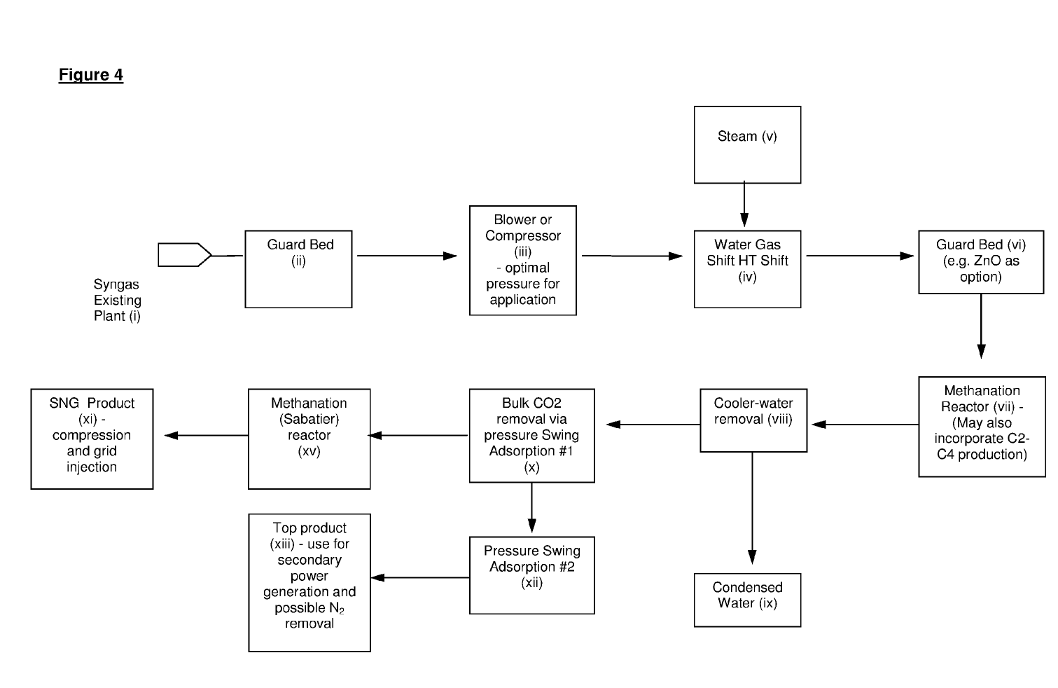

FIG. 4 shows a similar process is shown to that of FIG. 3, but in this case the methane-containing gas produced by the first pressure swing adsorption unit (x) is passed to a final methanation (Sabatier) reactor (xv) prior to injection into a gas grid.

The invention will now be described with reference to the following non-limiting examples.

### Example 1

A series of bench scale tests was carried out to demonstrate that high conversion of the reactant gases can be achieved over an extended period at low (e.g. less than 2 Bar) pressures and high CO and CO₂ partial pressures. A secondary objective of the test work was to demonstrate the ignition (light-off) temperature of the reaction as the ability to manage the catalytic reactor will be dependent on the temperature profile across the unit. A series of 3×8 hour tests runs were carried out and the feed and product gas analysis is summarised in Tables 1 and 2.

TABLE 1
Table of reaction conditions used for Methanation runs, M-17 to M-20 as reported

| | | | | | | System | | Exotherm |
|---|---|---|---|---|---|---|---|---|
| Run | | | Furnace | Steam | Conversion | Pressure | | Light-off |
| No | Catalyst | Gas feed | ° C. | % v/v | CO % | barg | GHSV | ° C. |
| M-17 | 3488/CT | N₂/H₂ | 400 | 0 | 0 | 2 | 2500 | — |
| | 300 | | | | | | | |
| M-18 | Ex M-17 | CO/CO₂/H₂ | 185 | 9 | 100 | 2 | 2040 | 210 |
| M-19 | Ex M-18 | CO/CO₂/H₂ | 150 | 9 | 100 | 2 | 2040 | 220 |
| M-20 | Ex M-19 | CO/CO₂/H₂ | 140 | 9 | 100 | 2 | 2040 | 227 |

1 Total catalyst+diluent volume=50 ml
2 GHSV calculated with respect to diluted volume
3 GHSV of 2040=1700 mls/min gas flow at STP
CATALYST: Johnson Matthey K11-4m pellets
Diluted 50:50 v/v with CT300 alumina 3 mm spheres

TABLE 2
Table of gas analyses for Methanation
runs, M-18 to M-20 as reported

| | | | | | Outlet | Catalyst |
|---|---|---|---|---|---|---|
| | CO | CO₂ | H₂ | CH₄ | gas flow | at max. |
| Run | content | content | content | content | ml/min | Exotherm/ |
| No | ppm | % v/v | % v/v | % v/v | STP | furnace ° C. |
| M-18 | 28 | 58.7 | 6.2 | 33.9 | 840 | 472/185 |
| M-19 | 45 | 58.3 | 6.0 | 34.1 | 857 | 468/150 |
| M-20 | 42 | 58.4 | 5.9 | 34.2 | 858 | 461/140 |

Values are averaged over each full run length when at constant catalyst temperature

1 CO & CO₂ are continuous Infra-Red analysis
2 CH₄ & H₂ are GC analyses
3 Analytical values+/−2%
4 Gas flow values+/−25 ml

The catalyst used for the series of test runs was a Johnson Matthey commercial methanation catalyst in pellet form—Katalco 11-4m containing 22% Ni (metallic basis). The catalyst was used in a 50% diluted form, with CT300 inert alumina 3 mm spheres used as the diluent.

The gas hourly space velocity (GHSV) used for the 3 activity test runs was 2040 with respect to steam free gas, with a gas feed of composition: CO=16.2%, CO₂=31.1%, H₂=52.7% which reflects a typical composition of gas that may be produced from the gasification/plasma treatment of a biomass feedstock. The outlet gas was analysed on stream, with continuous CO and CO₂ analysis and intermittent CH₄ analyses. Steam at 9% v/v was added to the inlet gas flow and the reactor operated at 2 Bar (absolute) pressure.

The key findings of the input and output results of this work are summarised in Tables 1 and 2. It is seen that a very high conversion of CO to methane was attained (˜100%) with very low residual levels of CO reported to be between 22 and 45 ppm. There was no indication of any significant reduction in the catalysts activity over the period of running the 3 trials. Shutting down the plant and restarting after an 8-hour run also did not appear to adversely impact the catalyst activity.

An important observation was that the light-off temperature for the catalyst, operating under the input conditions given in Tables 1 and 2, was between 210-230° C. This should allow flexibility of the reactor design which can therefore be operated under isothermal or adiabatic conditions (or a combination for reactors operating in series). Moreover, subcritical cooling of the reactor may be practiced allowing high heat removal efficiency from the reactor zone, which would permit operating at least part of the reactor vessel train under isothermal or quasi-isothermal conditions. A further observation was that the methanation reaction was kinetically fast which should limit the size of reactor required even when operating under relatively low pressures.

The foregoing detailed description has been provided by way of explanation and illustration, and is not intended to limit the scope of the appended claims. Many variations in the presently preferred embodiments illustrated herein will be apparent to one of ordinary skill in the art and remain within the scope of the appended claims and their equivalents.

1. A process for producing a substitute natural gas, the process comprising the steps of:
providing a synthesis gas comprising hydrogen and carbon monoxide;
subjecting the synthesis gas to a water-gas-shift reaction to increase the ratio of hydrogen to carbon monoxide thereby forming a hydrogen-enriched synthesis gas;
subjecting the hydrogen-enriched synthesis gas to a methanation reaction to convert at least a portion of the gas into methane thereby forming a methane-enriched gas; and
recovering from the methane-enriched gas a methane-containing gas having a Wobbe number of from 43 to 57 MJ/m³.

2. (canceled)

3. The process according to claim 1, wherein the methane-containing gas has a Wobbe number of from 45 to 55 MJ/M3.

4. The process according to claim 1, wherein at least a portion of the hydrogen-enriched synthesis gas is subjected to an alkane and/or alkene formation reaction to convert at least a portion of the gas into C2 and/or C3 and/or C4 alkanes/alkenes.

5. The process according to claim 1, wherein the ratio of hydrogen to carbon monoxide is increased to about 3:1 or higher.

6. The process according to claim 1, wherein the pressure of the synthesis gas during the water-gas-shift reaction and/or the gas during the methanation reaction and/or the gas during the alkane/alkene formation reaction is from 1 to 8 bar.

7. (canceled)

8. The process according to claim 4, wherein the step of subjecting the hydrogen-enriched synthesis gas to a methanation reaction and the step of subjecting the hydrogen-enriched synthesis gas to an alkane and/or alkene formation reaction are conducted in the same reaction vessel with multiple catalysts.

9. (canceled)

10. The process according to claim 1, wherein the methanation and/or alkane/alkene formation reaction is conducted at a temperature of from 200 to 450° C.

11. The process according to claim 1, wherein the methane-containing gas is recovered using pressure swing adsorption.

12. (canceled)

13. The process according to claim 1, wherein the methane-containing gas is recovered by removal of nitrogen from the methane-enriched gas.

14. (canceled)

15. The process of claim 14, further comprising using the secondary fuel gas in a gas turbine or gas engine.

16. (canceled)

17. The process according to claim 1, the method further comprising a step of recovering or removal of carbon dioxide from the synthesis gas after subjecting the synthesis gas to the methanation reaction.

18. The process according to claim 17, wherein the majority of the carbon dioxide is removed from the synthesis gas using pressure swing absorption prior to subjecting the synthesis gas to the Sabatier reaction.

19. The process according to claim 18, further comprising subjecting the synthesis gas to the Sabatier reaction for removal of the carbon dioxide therefrom.

20. The process according to claim 1, wherein the synthesis gas is produced by the gasification and/or plasma treatment of a feedstock material.

21. The process according to claim 20, wherein the feedstock is a waste material and/or comprises biomass.

22. The process according to claim 1, wherein the water-gas-shift reaction and/or the methanation reaction is carried out in a single step.

23. The process according to claim 1, wherein the synthesis gas is produced in a waste treatment process comprising:
(i) a gasification step comprising treating the waste in a gasification unit in the presence of oxygen and steam to produce an offgas and a non-airborne, solid char material; and
(ii) a plasma treatment step comprising subjecting the offgas and the non-airborne, solid char material to a plasma treatment in a plasma treatment unit in the presence of oxygen and, optionally, steam, wherein the plasma treatment unit is separate from the gasification unit.

24. The process according to claim 1 wherein the synthesis gas is produced by:
(i) thermally treating a feedstock material to produce a synthesis gas; and
(ii) plasma-treating the synthesis gas in a plasma treatment unit in the presence of additional carbon dioxide to produce a refined synthesis gas,
wherein the additional carbon dioxide is added to the feedstock material during the thermal treatment and/or to the synthesis gas before plasma treatment and/or introduced in the plasma treatment unit.

25. The process according to claim 1, the process further comprising combusting the substitute natural gas as a fuel, optionally in combination with at least a portion of natural gas.

26. (canceled)
